(19) United States
(12) Patent Application Publication
(10) Pub. No.: US 2026/0090843 A1
(43) Pub. Date: Apr. 2, 2026

(54) APPARATUS AND METHOD FOR MONITORING AND/OR CONTROLLING AN ADVANCE OF A MEDICAL INSTRUMENT, COMPUTER PROGRAM, AND DATA CARRIER

(71) Applicant: Siemens Healthineers AG, Forchheim (DE)

(72) Inventor: Marcus Pfister, Bubenreuth (DE)

(21) Appl. No.: 19/345,149

(22) Filed: Sep. 30, 2025

(30) Foreign Application Priority Data
Sep. 30, 2024 (DE) 10 2024 209 503.6

(51) Int. Cl. A61B 34/20; A61B 17/00; A61B 90/30

(52) U.S. Cl. CPC A61B 34/20; A61B 17/00234; A61B 90/30; A61B 2017/00292; A61B 2034/2055; A61B 2034/2061; A61B 2090/306

(57) ABSTRACT

An apparatus for monitoring and/or controlling an advance of a medical instrument through a vascular system of a patient includes a processing device configured, at at least one check time, to detect or receive speed information relating to a speed of advance of the medical instrument. The processing device is configured to detect or receive force information relating to a force acting on the medical instrument, evaluate a triggering condition, fulfillment of which depends on both the speed information and the force information, and when the triggering condition is fulfilled, actuate an indicating device to output an indication to a user of the apparatus and/or actuate an actuator system moving the medical instrument to stop the advance of the medical instrument.

This application claims the benefit of German Patent Application No. DE 10 2024 209 503.6, filed on Sep. 30, 2024, which is hereby incorporated by reference in its entirety.

### BACKGROUND

The present embodiments relate to an apparatus for monitoring and/or controlling an advance of a medical instrument through a vascular system of a patient, a method for monitoring an advance of a medical instrument, a computer program, and a data carrier.

Minimally invasive procedures are performed using instruments that are guided (e.g., by a catheter or a probe) through the vascular system of the patient to a relevant site in order to obtain diagnostically relevant information and/or carry out a local intervention. In individual cases, an unexpected characteristic of the vascular system and/or incorrect guidance of the instrument may result in an undesirable perforation of a vessel wall. If such a perforation is detected in good time, adverse consequences for the patient may be avoided or minimized by promptly initiating countermeasures (e.g., by local cauterization).

Such a perforation of the vessel wall may be detected by experienced medical personnel using fluoroscopy, for example (e.g., continuous monitoring of the procedure by X-ray imaging, such as using a C-arm angiography system). As an alternative or in addition to fluoroscopic monitoring of the intervention, it is possible to determine the shape of the instrument inside the body using approaches that analyze light reflections at Bragg gratings in a plurality of glass fibers integrated into the instrument, in order to superimpose the instrument having the determined shape on a previously determined image of the vascular system. Such a system is presented, for example, on the website https://shapesensing.com/.

If the instrument is advanced manually, perforation of the vessel wall may potentially also be detected directly based on the haptic feedback from the forces occurring in the process. However, in the case of an automated advance (e.g., by a treatment robot or in the field of telemedicine), there is often no haptic feedback, or at least no haptic feedback that is sufficiently detailed for this purpose.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a user may be better assisted in quickly detecting and/or preventing perforation of a vessel wall by a medical instrument.

In one embodiment, an apparatus for monitoring and/or controlling an advance of a medical instrument through a vascular system of a patient is provided. At at least one check time, a processing device of the apparatus is configured in each case to: detect speed information relating to a speed of advance of the medical instrument or receive the speed information from a source external to the apparatus; detect force information relating to a force acting on the medical instrument or receive the force information from the or another source external to the apparatus; evaluate a triggering condition, fulfillment of which depends on both the speed information and the force information; and when the triggering condition is fulfilled, actuate an indicating device to output an indication to a user of the apparatus and/or actuate an actuator system moving the medical instrument to stop its advance.

Analysis of the force information enables, for example, detection of a marked increase in an axial force acting on a distal end of the medical instrument when the distal end of the medical instrument abuts a vessel wall and threatens to perforate the vessel wall. Detecting this situation may, if an indication is issued in good time or the actuator system is actuated so as to stop the advance, make it possible to avoid perforation of the vessel wall entirely.

However, because this increase in force occurs only briefly before or during perforation of the vessel wall and therefore, taken on its own, does not in all cases permit robust detection of perforation, in addition or alternatively, use may be made of the fact that, after perforation of the vessel wall, further advance of the medical instrument, depending on the surroundings of the vessel, results in almost all cases in significantly higher or significantly lower counter-forces opposing the advance than those occurring during advance inside the vessel.

Thus, the triggering condition may be fulfilled, for example, whenever the force information deviates sufficiently strongly from an expected value for the counter-force occurring during advance within the vascular system. The expected magnitude of the force acting on the medical instrument (e.g., the magnitude of the force acting in the axial direction, such as along the medical instrument or the vessel) counter to the advance depends strongly on the speed of advance. Therefore, according to the present embodiments, the speed information is additionally taken into account as part of the triggering condition in order to improve the robustness of detection.

Thus, the approach of the present embodiments provides a robust method of detecting a prior perforation of the vessel wall by the medical instrument, and of alerting the user of the apparatus, such as a physician performing the procedure, to this vessel wall injury so that the user may quickly and reliably take countermeasures or prevent further advance in order to minimize damage to the vascular system or to the surrounding tissue. It may even be possible to prevent perforation of the vessel wall by the medical instrument by timely detection of an increased force due to wall contact.

The processing device may, for example, be configured to actuate the indicating device only when the triggering condition is fulfilled, specifically to provide a particular indication. Additionally or alternatively, the processing device may be configured to actuate the actuator system when the triggering condition is fulfilled (e.g., to do so exclusively when the triggering condition is fulfilled), such that any advance of the medical instrument is stopped even if a request to advance the medical instrument further is received via an input interface, such as a joystick or a telemedical data connection.

The speed information may be provided, for example, by a robot moving the medical instrument, and the force information may be provided by a measuring device separate from the robot. In the case of robotic advance, the speed information may be available as control information for an actuator system or may be determined based on such control information. For example, when the medical instrument is advanced manually, but also when the medical instrument is advanced by a robot, the speed of advance may alternatively or additionally be detected by a suitable sensor system.

The processing device may be configured to determine, from the speed specified by the speed information and using a predefined assignment rule, an expected value for the force acting on the medical instrument. The triggering condition is fulfilled when a magnitude of the force acting on the medical instrument as described by the force information falls below the expected value by a predefined deviation value.

Thus, a known or previously determined relationship between the speed of advance and the force expected at that speed during advance of the medical instrument within the vascular system may be used to detect when unexpectedly low forces occur, indicating that the vessel wall has been perforated and the medical instrument is therefore being advanced into a region outside the vascular system where lower forces are required for advance. Such a region may be, for example, a cavity in the body of the patient that adjoins the vascular system.

In a simple example, the assignment rule may describe a linear relationship between the speed specified by the speed information and the expected value for the force acting on the medical instrument. However, more complex relationships are also possible, which may be modeled, for example, by a look-up table as the assignment rule (e.g., with linear interpolation).

The processing device may additionally or alternatively be configured to determine, using the or a predefined assignment rule, the or an expected value for the force acting on the medical instrument from the speed specified by the speed information. The triggering condition is fulfilled when a magnitude of the force acting on the medical instrument as described by the force information exceeds the expected value by the or a predefined deviation value.

Thus, the occurrence of unexpectedly high forces may also be detected, for example, if the vessel wall has already been perforated and the medical instrument is being advanced into tissue adjoining the vascular system. Detecting unexpectedly high forces is also advantageous for detecting that the medical instrument is being pressed against the vessel wall and thereby potentially preventing perforation of the vessel wall by a timely warning or corresponding control of the actuator system.

The processing device may be configured to: detect, at a plurality of reference times prior to the check time or at least one of the check times, a respective reference value pair consisting of a reference speed value that relates to a speed of advance of the medical instrument at the respective reference time, and a reference force value that relates to a force acting on the medical instrument at the respective reference time, or receive these values from the or another source external to the apparatus; and determine, based on these reference value pairs, the predetermined assignment rule and/or the predetermined deviation value, or a predetermined relationship between the deviation value and the speed information.

Thus, the expected value of the force, or a permissible degree of deviation from this expected value, may be based on empirical values collected during a previous advance of the medical instrument (e.g., in the vascular system of the same patient, specifically during the same intervention). For example, a user input may signal the start of an intervention or an initial advance of the medical instrument within the vascular system, and the processing device may be configured to acquire or receive reference value pairs from that point in time (e.g., periodically at short intervals).

The reference value pairs may be statistically analyzed to determine the assignment rule and/or the deviation value or the predefined relationship. For example, a look-up table may be determined as an assignment rule and then used, directly or interpolated, to determine the expected value. In this case, generally in relation to the statistical analysis of the reference value pairs, binning may be performed with respect to the reference speed values. For example, for a plurality of speed intervals, a mean value of all the reference force values assigned to reference speed values within the respective speed interval is determined and assigned as the expected value for the mean speed of that speed interval.

The assignment rule may assign to all speeds in the respective speed interval, as a deviation value, for example, the standard deviation or a predefined multiple of the standard deviation of those reference force values that are assigned to reference speed values within the speed interval. Alternatively, this standard deviation or multiple thereof may also be assigned only to the mean speed in the respective interval, and the resulting look-up table may be interpolated for other speeds. As a further alternative, the same deviation value may also be used for all speed information. The deviation value may, for example, be selected as the mean of these standard deviations or a predefined multiple of this mean.

Alternatively, the reference value pairs may be used, for example, to determine parameters of an analytical relationship, such as a linear relationship, between the speed information and the expected value by a fit, or by regression analysis. The assignment rule, or the specified deviation value, may then be determined, for example, as the standard error of the regression or as a predefined multiple thereof.

The processing device may be configured such that, in cases where the triggering condition is not fulfilled at the respective check time, the processing device consistently, or when an additional condition is fulfilled, uses the force information determined for that check time as the reference force value of the reference value pair for the reference time corresponding to the check time and the speed information as the reference speed value of this reference value pair.

Thus, in this case (e.g., if there appears to be no perforation of the vessel wall), the force and speed information already determined at the check time for evaluating the triggering condition may be used to provide an additional reference value pair for subsequent check times. In other words, the assignment rule and, optionally, also the deviation value, or the predefined relationship between the deviation value and the speed information, may be adjusted quasi-continuously during the advance in order to obtain an increasingly accurate profile of the forces to be expected in the course of the advance.

The apparatus may include the medical instrument. The medical instrument has, in a distal section that is configured to be located within the vascular system during the advance of the medical instrument through the vascular system, at least one sensor component by which a force component of the force acting on the distal section in a longitudinal direction of the medical instrument may be detected as the force information or as part of the force information. The direction extends, during the advance of the medical instrument in the vascular system, along the vessel in which the distal section is located.

The sensor components may thus be used to measure, for example, the force or force component that counteracts a further advance of the distal end of the medical instrument. Within the vascular system, this force may result primarily from the displacement of the blood located in front of the medical instrument in the vascular system. Depending on the specific design of the sensor component, friction between the medical instrument and the vessel wall in the region of the distal section may also contribute to the measured force component.

If, for example, the medical instrument is advanced into a cavity in the body after perforating a vessel wall, the measured force component may be significantly lower than within the vascular system. Conversely, this force component may increase significantly compared to movement within the vascular system if, for example, the medical instrument is advanced through adjoining tissue after perforation of a vessel wall of the vascular system.

In addition or as an alternative to using such a sensor component, the total force required to advance the medical instrument or a quantity correlated therewith (e.g., a motor current of the actuator system performing the advance) may also be measured. Measuring only the total force required for advance may reduce the technical complexity for implementing the apparatus according to the present embodiments, since measurements outside the body of the patient are technically easier to implement than force measurements inside the body. However, by using sensor components arranged in the distal section of the medical instrument, an impending or already occurred vessel wall injury may potentially be detected more robustly, since exclusive analysis of the total force required for advance may include significant contributions from friction of the medical instrument with the vascular system over the entire length of the medical instrument. Thus, the forces that actually change during or after perforation of a vessel wall make only a minor contribution to the measured variable when the total force required for advance is evaluated. This may result in a significantly lower signal-to-noise ratio than when the sensor component in the distal section is used.

The medical instrument may be or include at least one optical fiber extending from the distal section to a proximal section of the medical instrument. The proximal section is configured to be disposed outside the patient when the medical instrument is being advanced through the vascular system. The sensor component is constituted by at least one Bragg grating forming part of the optical fiber. A light source of the apparatus is configured to irradiate light into the respective optical fiber. A sensor device of the apparatus is configured to detect a light component reflected by the respective Bragg grating and to provide sensor data relating to this light component to the processing device. The processing device is configured to determine the force information as a function of this sensor data.

A Bragg grating may be formed, for example, by periodically varying the refractive index of the fiber material in the longitudinal direction of the fiber and thus of the medical instrument. Such a variation may be achieved, for example, by locally heating a plastic fiber using a laser interference pattern. Such a Bragg grating essentially backscatters only light of a specific wavelength predefined by the periodicity of the Bragg grating.

A force acting on the distal section compresses the Bragg grating due to the elasticity of the instrument or of the optical fiber, thereby shifting the wavelength of the backscattered light toward shorter wavelengths. By irradiating light into the optical fiber and determining the wavelength of the backscattered light, the force acting on the distal section may therefore be determined, such that the Bragg grating located there functions as a sensor component of a fiber optic sensor.

Using such a fiber optic sensor to determine the force information is advantageous, since an optical fiber with one or more Bragg gratings requires only a small installation space perpendicular to the direction of advance of the instrument. Compared to an electrical sensor, such a fiber optic sensor is also less susceptible to interference (e.g., when X-ray imaging is used during the intervention). Especially when a plurality of optical fibers each with a plurality of Bragg gratings are used, such a sensor system may also be employed to determine further information about the instrument (e.g., to detect its current shape).

In addition or alternatively, the force component may be provided, for example, by a pressure sensor in the distal section (e.g., a pressure sensor based on piezoelectric or film capacitor technology).

The respective optical fiber may be configured as a Bragg grating in a measuring section extending from the distal section of the medical instrument over at least 20% or at least 50% of the length of the medical instrument between the distal and proximal sections. The Bragg grating is formed either continuously or in a plurality of spaced-apart subsections.

In the case where the measuring section is configured as a Bragg grating in a plurality of spaced-apart subsections, for example, at least three or at least five such subsections may be present and/or at least one of these subsections may adjoin the end of the measuring section facing away from the distal section of the instrument.

If a plurality of Bragg gratings or a continuous Bragg grating are used in the measuring section, a curvature of the measuring section, for example, results in different degrees of compression and/or expansion of the different Bragg gratings or of different sections of the continuous Bragg grating, so that, depending on the magnitude of the local compression or expansion, a light component of a different wavelength is reflected at different positions of the measuring section. By evaluating the spectral component of the light for which reflected light is received after a given propagation time or propagation times (e.g., if the light source is operated in a pulsed manner), information about the deformation of the optical fiber and thus about the shape of the instrument or the forces acting on the instrument may be determined over the entire length of the measuring section. In principle, the measuring section may extend over the entire length of the instrument or of the optical fiber.

The medical instrument may include at least three of the optical fibers that are arranged at least in a plurality of measuring planes within the measuring section, in each case spaced apart from one another perpendicularly to the longitudinal direction of at least one of the fibers. The processing device is configured to determine, based on the sensor data, a local curvature of the medical instrument in the region of the respective measuring plane and thus information relating to the shape of the medical instrument.

A curvature of the instrument in the region of the respective measuring plane causes the different optical fibers to be stretched or compressed to different extents, depending on the direction and degree of curvature. The respective stretching or compression may be detected and quantified by a respective Bragg grating in the region of the respective measuring plane or a respective section of a continuous Bragg grating located in the region of the respective measuring plane, so that, if the arrangement of the optical fibers in the measuring plane is known, the local curvature in the region of the respective measuring plane may be determined, whereby, if the optical fibers are connected to the instrument in a substantially rigid manner, the shape of the instrument in the measuring section may be determined. This may be used, for example, to visualize the medical instrument for the user. The shape determined based on the sensor data may, for example, be displayed superimposed on a previously determined image data set of the vascular system.

As already explained in the introduction, determining the shape of a medical instrument based on fiber optic sensors is known per se. However, it has been recognized that fiber optic sensors that are used, for example, to determine the current shape of the medical instrument may additionally be used according to the present embodiments to determine the force information analyzed in the context of the triggering condition, thereby making it possible, for example, to detect an imminent or already occurred perforation of a vessel wall by the medical instrument. Thus, the apparatus according to the present embodiments may be implemented in a particularly simple manner if, for example, fiber optic sensors are to be used in any case to detect the shape of the instrument. The speed information may already be known, particularly in the case of automatic advance of the instrument, so that the apparatus according to the present embodiments may be implemented with minimal technical complexity based on already known or used resources (e.g., by a suitable software modification).

The processing device may be configured to check fulfillment of the triggering condition periodically at check time intervals of less than 50 ms or less than 10 ms. For example, the check times may be spaced less than 5 ms apart (e.g., one millisecond apart). The force information and the speed information may be redetermined for each check time. Using the approaches explained above for determining this information, this may easily be accomplished with the stated check time intervals or the sampling frequency corresponding to this time interval. This makes it possible to react essentially without delay to a perforation of the vessel wall that has already occurred or is imminent.

In addition to the apparatus according to the present embodiments, the present embodiments relate to a computer-implemented method for monitoring an advance of a medical instrument within a vascular system of a patient, including the acts, performed at at least one check time, of: receiving or determining speed information relating to the speed of advance of the medical instrument; receiving or determining force information relating to a force acting on the medical instrument; evaluating a triggering condition, fulfillment of which depends on both the speed information and the force information; and actuating an indicating device to output an indication to a user when the triggering condition is fulfilled, and/or determining control information for an actuator system for moving the medical instrument depending on whether the triggering condition is fulfilled. The control information determined when the triggering condition is fulfilled relates to stopping the advance of the medical instrument.

The advance of the medical instrument within the vascular system and/or the determination of the speed or force information may be carried out separately from the method according to the present embodiments. Alternatively, at least one of these acts or all of these acts may also be incorporated into the method.

The apparatus according to the present embodiments (e.g., its processing device) may be configured, for example, to carry out the computer-implemented method according to the present embodiments. Accordingly, the computer-implemented method of the present embodiments may be implemented and optionally developed by the features of the apparatus according to the present embodiments as explained above. If the features and embodiments explained above in relation to the apparatus according to the present embodiments are transferred to the computer-implemented method according to the present embodiments, the advantages discussed above in this respect are also achieved. In general, the computer-implemented method according to the present embodiments may thus be developed using the features described for the apparatus according to the present embodiments and vice versa, whereby the advantages explained for the respective feature may be achieved.

The present embodiments further relate to a computer program with instructions that, when executed on a data processing apparatus, are configured to carry out the computer-implemented method according to the present embodiments and/or to implement the processing device of the apparatus according to the present embodiments. The data processing apparatus may be configured together with other components of the apparatus (e.g., with an actuator system for advancing the medical instrument and/or a user interface for the actuator system), or the data processing apparatus may also be configured separately from the other components of the apparatus described above (e.g., as a workstation computer, server, or cloud solution).

The present embodiments further relate to a data carrier (e.g., a non-transitory computer-readable storage medium) containing the computer program according to the present embodiments.

Independent of the grammatical term usage, individuals with male, female, or other gender identities are included within the term.

### BRIEF DESCRIPTION OF THE DRAWINGS

Further advantages and details of the invention will emerge from the following example embodiments and the accompanying schematic drawings, in which:

FIG. 1 shows an example embodiment of an apparatus;

FIG. 2 shows a flow diagram of an example embodiment of a method; and

FIG. 3 shows a sectional view of the medical instrument 2 used in FIG. 1.

### DETAILED DESCRIPTION

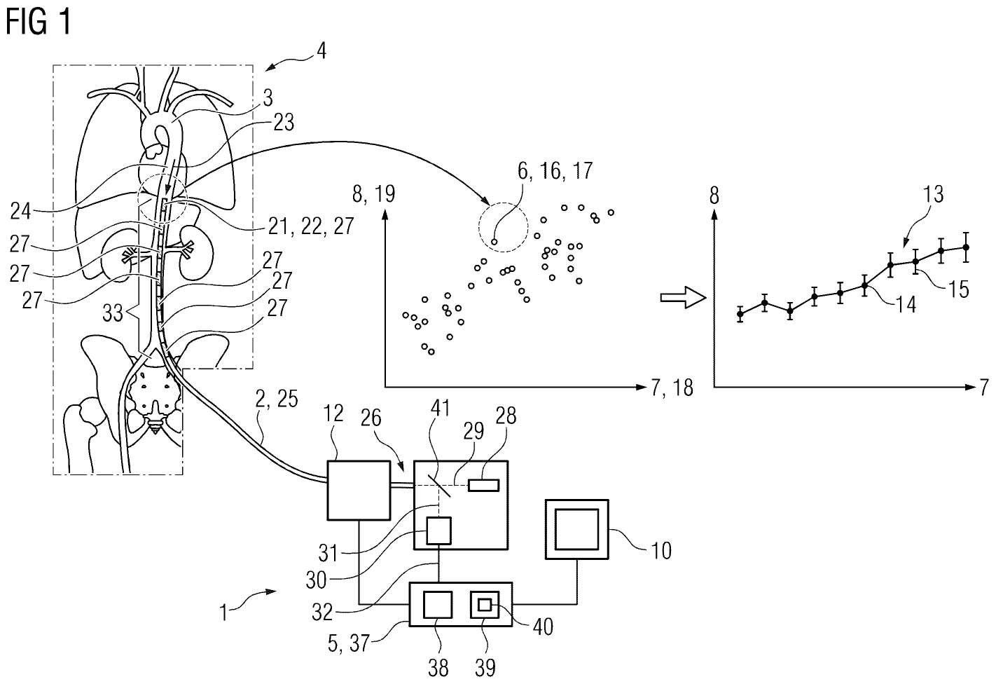

FIG. 1 shows an apparatus for monitoring and controlling an advance of a medical instrument 2 through a vascular system 3 of a patient 4. In the use situation shown in FIG. 1, the medical instrument 2 (e.g., a catheter) has already been inserted some way into the vascular system 3 of the patient 4. The vascular system 3 is accessed via the groin of the patient 4. In the example, the medical instrument 2 is advanced through the vascular system 3 by an actuator system 12 of the apparatus 1 (e.g., by being unwound from a roll (not shown)). The advance may be controlled, for example, by a local operating element (not shown), such as a joystick, or also by telemedicine.

A processing device 5 of the apparatus 1 is configured to detect, periodically at a plurality of check times 6 (e.g., at intervals of 1 ms), speed information 7 relating to a speed of advance of the medical instrument 2 and force information 8 relating to a force acting on the medical instrument 2. Possibilities for detecting these variables will be explained later. As an alternative to detection of these variables by the processing device, these variables may also be detected by a separate source or by a plurality of separate sources (e.g., by a suitable sensor system and/or robotics provided separately from the apparatus 1).

The processing device also checks at the respective check time whether a triggering condition 9 is fulfilled, fulfillment of which depends on both the speed information 7 and the force information 8. In the example, if the triggering condition 9 is fulfilled, an indicating device 10 (e.g., a monitor) is controlled to output an indication 11 to a user of the apparatus 1, and the actuator system 12 moving the medical instrument 2 is actuated to stop the advance of the medical instrument 2, since in this case it is assumed that a vessel wall of the vascular system 3 has been perforated by the end of the instrument 2 facing away from the actuator system 12.

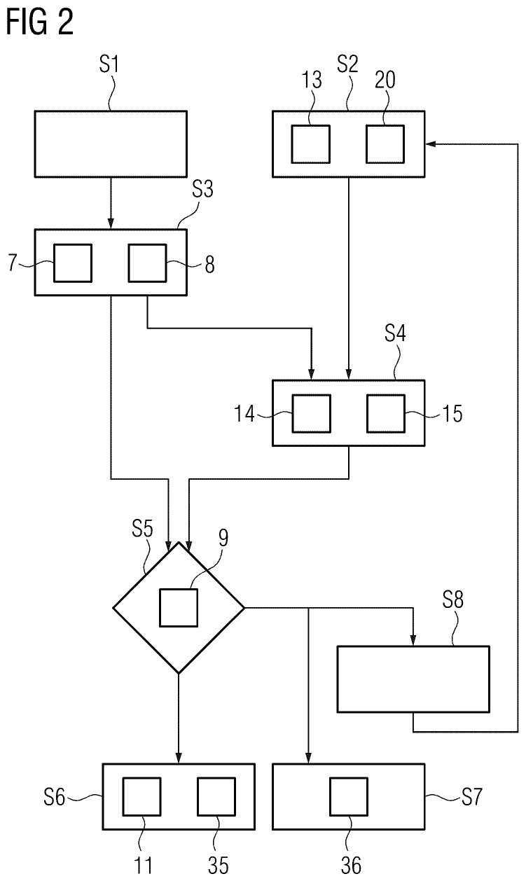

Further details of the apparatus 1 will be explained below with additional reference to the flow diagram shown in FIG. 2.

There, in act S1, the instrument 2 is first introduced into the vascular system 3. In act S2, an initial assignment rule 13 is predefined, which assigns to different values of the speed information a respective expected value 14 for the force acting on the medical instrument 2. In addition, an initial relationship 20 between the permissible deviation value 15 and the speed information 7 is predefined.

An example of such an assignment rule 13 and of resulting deviation values 15 for different speed information 7 is shown in the right-hand graph in FIG. 1. The initial assignment rule 13 and the initial relationship 20 may be based on empirical values or on monitoring of previous interventions. This information may, for example, be specific to a particular type of intervention and/or a particular person controlling the intervention. As will be explained later, the assignment rule 13 and the relationship 20 may be dynamically adjusted in the course of monitoring the advance of the medical instrument 2 in order to adapt the force-speed profile predefined by these variables to the specific patient 4 and the specific intervention (e.g., to the section of the vascular system 3 used to introduce the medical instrument 2).

In act S3, the speed information 7 and the force information 8 for the respective check time 6 are then determined. In the example, each point on the scatter plot shown on the left in FIG. 1 corresponds to a value pair for a respective check time 6.

Control information for the actuator system 12, which in the example is provided by the processing device 5 itself (e.g., the control information 36 provided in the previous repetition in act S7 in the case of repetitions of the method) may be used directly as speed information.

In the example, a fiber optic sensor is used to determine the force information 8. For this purpose, the medical instrument 2 includes at least one optical fiber 25 that extends in the longitudinal direction of the instrument 2 and, in the example, includes a plurality of Bragg gratings in a measuring section 33. Light 29 is coupled into the optical fiber 25 by a light source 28 of the apparatus 1. Each of the Bragg gratings 27 reflects only a light component 31 in a narrow frequency band predefined by the grating constant of the respective Bragg grating 27. The respective reflected light component 31 is deflected onto the sensor 30 by the semi-transparent mirror 41. If the light source 28 is operated in pulsed mode, for example, the different propagation times of the light components 31 reflected by different Bragg gratings may be used to determine from which Bragg grating 27 a light component 31 is currently being received. Spectral analysis of the respective light component 31 indicates the actual grating constant of the respective Bragg grating 27, whereby deformation of the optical fiber 25 in the region of the respective Bragg grating 27 may be quantified. Based on the known mechanical properties of the fiber 25 of the instrument 2, the deformation of the optical fiber 25 may also be used to infer a force input in the region of the respective Bragg grating 27.

As will be explained later with reference to act S8, the resulting sensor data 32 may also be used to obtain information about the current shape of the instrument.

In act S3, only the reflection by the Bragg grating arranged at the distal end or in the distal section 21 of the medical instrument 2 is initially taken into account. A deformation of this Bragg grating 27 and thus a shift in the frequency of the light component 32 reflected by this Bragg grating 27 is determined, at least approximately, by a force component 23 of the force acting on the distal section 21 in a longitudinal direction of the medical instrument 2 (e.g., by that force component that counteracts an advance of the medical instrument 2). This Bragg grating 27 thus functions as a sensor component 22 by which the desired force information 8 may be determined.

In act S4, based on the speed information 7 determined in act S3, an expected value 14 for the magnitude of the force component 23 is determined by the assignment rule 13, and a deviation value 15 is determined using the relationship 20.

In act S5, it is then checked whether the magnitude of the force acting on the medical instrument, as described by the force information 8, exceeds or falls below the expected value 14 by a predetermined deviation value 15 (e.g., deviates sufficiently from a value expected for an advance within the vascular system 3).

If this is the case, as already explained in the general part of the description, this indicates that the distal end of the medical instrument 2 is at risk of perforating a vessel wall of the vascular system 3 or has already perforated the vessel wall of the vascular system 3. In this case, in act S6, control information 35 is therefore provided to the actuator system 12, which stops the advance of the medical instrument 2. In addition, the indicating device 10 is actuated to output an indication 11 in order to enable a user to respond promptly to the possible vessel wall injury. For example, the user may retract the instrument 2 and cauterize this perforation.

If the triggering condition 9 is not fulfilled, the advance of the medical instrument 2 may be continued by outputting the control information 36 to the actuator system 12. Since in this case it is assumed that the advance has taken place within the vascular system at the check time 6, the determined force information 8 may be temporarily stored as a reference force value 19 and the determined speed information 7 as a reference speed value 18 of a reference value pair 17. As soon as a sufficient number of reference value pairs 17 is available, an updated assignment rule 13 and an updated relationship 20 between the deviation value 15 and the speed information 7 may be determined based on the reference value pairs 17 (e.g., after each check time 6 or after a predetermined number of check times 6).

For example, each of the points shown in the left-hand plot in FIG. 1 may correspond to such a reference value pair 17. For the scatter plot shown, as already explained in detail in the general part of the description, a fit of a predetermined function or binning of the reference value pairs may then be carried out to determine mean values and thereby define the updated assignment rule 13. As likewise already explained in the general part, the updated relationship 20 may be determined by the fit error, the standard error of the regression, or a standard deviation in binning.

Optionally, for each check time 6 or, for example, after a predetermined number of check times 6, in act S8, a shape of the medical instrument 2 may be determined in a manner known per se (e.g., in order to superimpose a synthetic representation of the medical instrument 2 onto a previously captured image of the vascular system 3 or of the patient 4 for display on the display device 10).

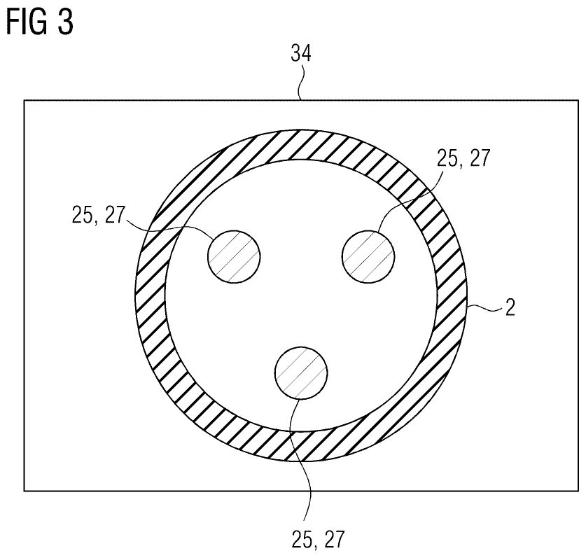

Since local curvatures in different directions and with different magnitudes are to be detected and quantified for this purpose, it is advantageous if the medical instrument 2 includes at least three of the optical fibers 25 that, as shown by way of example in FIG. 3, are arranged in a plurality of measuring planes 34 within the measuring section 33, in each case spaced apart from one another in a plane perpendicular to the longitudinal direction of the fibers 25. As already explained in the general part, a curvature of the medical instrument 2 in the region of such a measuring plane 34 causes the various light-transmissive fibers 25 to be compressed or stretched to different extents. If a Bragg grating 27 is located in each of the fibers in the region of the measuring plane 34, the different degrees of compression or stretching may be quantified, making it possible to infer the shape of the medical instrument 2 from its known mechanical properties.

In the example shown in FIG. 1, the processing device 5 is implemented by a freely programmable data processing device 37 (e.g., a workstation computer or a server), the processor 38 of which executes a computer program 40 implementing the processing steps described. The computer program 40 is stored on a data carrier 39.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An apparatus for monitoring, controlling, or monitoring and controlling an advance of a medical instrument through a vascular system of a patient, the apparatus comprising:
a processing device, wherein, at at least one check time, the processing device is configured in each case to:
detect speed information relating to a speed of advance of the medical instrument or receive the speed information from a source external to the apparatus;
detect force information relating to a force acting on the medical instrument or receive the force information from the source external to the apparatus or another source external to the apparatus;
evaluate a triggering condition, fulfillment of which depends on both the speed information and the force information; and
when the triggering condition is fulfilled:
actuate an indicating device to output an indication to a user of the apparatus;
actuate an actuator system moving the medical instrument to stop the advance of the medical instrument; or
a combination thereof.

2. The apparatus of claim 1, wherein the processing device is further configured to determine, using a predefined assignment rule, an expected value for the force acting on the medical instrument from the speed specified by the speed information,
wherein the triggering condition is fulfilled when a magnitude of the force acting on the medical instrument, as described by the force information, falls below the expected value by a predefined deviation value.

3. The apparatus of claim 1, wherein the processing device is further configured to determine, using a predefined assignment rule, an expected value for the force acting on the medical instrument from the speed specified by the speed information, and
wherein the triggering condition is fulfilled when a magnitude of the force acting on the medical instrument, as described by the force information, exceeds the expected value by a predefined deviation value.

4. The apparatus of claim 2, wherein, at a plurality of reference times prior to a check time of the at least one check time, the processing device is further configured to:
detect, or receive from the source external to the apparatus or the other source external to the apparatus, a respective reference value pair consisting of a reference speed value relating to a speed of advance of the medical instrument at the respective reference time, and a reference force value relating to a force acting on the medical instrument at the respective reference time; and
determine the predefined assignment rule, the predefined deviation value, or the predefined assignment rule and the predefined deviation value, or a predefined relationship between the predefined deviation value and the speed information based on the reference value pairs.

5. The apparatus of claim 3, wherein, at a plurality of reference times prior to a check time of the at least one check time, the processing device is further configured to:
detect, or receive from the source external to the apparatus or the other source external to the apparatus, a respective reference value pair consisting of a reference speed value relating to a speed of advance of the medical instrument at the respective reference time, and a reference force value relating to a force acting on the medical instrument at the respective reference time; and
determine the predefined assignment rule, the predefined deviation value, or the predefined assignment rule and the predefined deviation value, or a predefined relationship between the predefined deviation value and the speed information based on the reference value pairs.

6. The apparatus of claim 4, wherein in cases in which the triggering condition is not fulfilled at the respective check time, the processing device is further configured to consistently, or when a supplementary condition is fulfilled, use the force information determined for the respective check time as the reference force value of the reference value pair for the reference time corresponding to the check time, and the speed information as the reference speed value of the reference value pair.

7. The apparatus of claim 1, further comprising the medical instrument,
wherein the medical instrument comprises, in a distal section that is configured to be located within the vascular system during the advance of the medical instrument through the vascular system, at least one sensor component configured to detect a force component of a force acting on the distal section in a longitudinal direction of the medical instrument as the force information or as part of the force information, the longitudinal direction of the medical instrument extending during the advance of the medical instrument in the vascular system along a vessel within which the distal section is located.

8. The apparatus of claim 7, wherein the medical instrument is or comprises at least one optical fiber extending from the distal section to a proximal section of the medical instrument, the proximal section being configured to be located outside the patient when the medical instrument is advanced through the vascular system,
wherein the sensor component is formed by at least one Bragg grating that forms part of the at least one optical fiber,
wherein a light source of the apparatus is configured to irradiate light into the respective optical fiber,
wherein a sensor device of the at least one sensor device is configured to:
detect a light component of the light reflected by the respective Bragg grating; and
provide sensor data relating to the light component to the processing device, and
wherein the processing device is further configured to determine the force information as a function of the sensor data.

9. The apparatus of claim 8, wherein the respective optical fiber is configured as a Bragg grating in a measuring section extending from the distal section of the medical instrument over at least 20% or at least 50% of the length of the medical instrument between the distal section and the proximal section, the Bragg grating being formed continuously or in a plurality of spaced-apart subsections.

10. The apparatus of claim 9, wherein the medical instrument comprises at least three optical fibers that are arranged, at least in a plurality of measuring planes within the measuring section, in each case spaced apart from one another in a direction perpendicular to the longitudinal direction of one or more of the at least three optical fibers, and
wherein the processing device is further configured to determine, as a function of the sensor data, a local curvature of the medical instrument in a region of the respective measuring plane and thus to determine shape information relating to the medical instrument.

11. The apparatus of claim 1, wherein the processing device is further configured to check fulfillment of the triggering condition periodically at check times that are less than 50 ms or less than 10 ms apart.

12. A computer-implemented method for monitoring an advance of a medical instrument within a vascular system of a patient, the computer-implemented method comprising:
at at least one check time:
receiving or determining speed information relating to a speed of advance of the medical instrument;
receiving or determining force information relating to a force acting on the medical instrument;
evaluating a triggering condition, fulfillment of which depends on both the speed information and the force information; and
actuating an indicating device to output an indication to a user when the triggering condition is fulfilled, determining control information for an actuator system for moving the medical instrument depending on whether the triggering condition is fulfilled, or a combination thereof,
wherein the control information determined when the triggering condition is fulfilled relates to stopping the advance of the medical instrument.

13. In a non-transitory computer-readable storage medium that stores instructions executable by one or more processors to monitor an advance of a medical instrument within a vascular system of a patient, the instructions comprising:
at at least one check time:
receiving or determining speed information relating to a speed of advance of the medical instrument;
receiving or determining force information relating to a force acting on the medical instrument;
evaluating a triggering condition, fulfillment of which depends on both the speed information and the force information; and
actuating an indicating device to output an indication to a user when the triggering condition is fulfilled, determining control information for an actuator system for moving the medical instrument depending on whether the triggering condition is fulfilled, or a combination thereof,
wherein the control information determined when the triggering condition is fulfilled relates to stopping the advance of the medical instrument.
